(19) United States
(12) Patent Application Publication
(10) Pub. No.: US 2006/0213759 A1
(43) Pub. Date: Sep. 28, 2006

(54) Pyrolysis apparatus and pyrolysis method

(75) Inventors: Frank Holzschuh, Besigheim (DE); Reinhold Prothmann, Esslingen (DE); Guenther Renz, Filderstadt (DE)

Correspondence Address: Lipsitz & McAllister, LLC, 755 MAIN STREET, MONROE, CT, 06468, US

(73) Assignee: Deutsches Zentrum fuer Luft- und Raumfahrt e.V., Koeln (DE)

(21) Appl. No.: 11/437,042

(22) Filed: May 19, 2006

Related U.S. Application Data
(63) Continuation of application No. 10/233,305, filed on Aug. 30, 2002.

(30) Foreign Application Priority Data
Sep. 5, 2001 (DE) 101 43 375

(51) Int. Cl. C07C 1/00

(52) U.S. Cl. 204/157.15

(57) ABSTRACT

A pyrolysis apparatus and pyrolysis method are provided. The pyrolysis apparatus comprises a microwave generator, a waveguide which is coupled to the microwave generator and in which a standing wave can be generated, and a fluid pipe, through which a fluid can be guided in a fluid guidance direction transverse to the direction of propagation of the standing wave. A pyrolysis cell, in which the fluid is acted upon by the standing electromagnetic wave, is formed in the fluid pipe. In order to achieve a high rate of degradation of molecules to be pyrolysed, the pyrolysis cell is limited in the direction of an outlet by a metal grid.

This application is a continuation of U.S. patent application Ser. No. 10/233,305 filed on Aug. 30, 2002, which claims the benefit of German patent application no. 101 43 375.1 filed on Sep. 5, 2001, each of which is incorporated herein and made a part hereof by reference.

### BACKGROUND OF THE INVENTION

The invention relates to a pyrolysis apparatus, comprising a microwave generator, a waveguide, which is coupled to the microwave generator and in which a standing wave can be generated, and a fluid pipe, through which a fluid can be guided in a fluid guidance direction transverse to the direction of propagation of the standing wave, wherein a pyrolysis cell, in which the fluid can be acted upon by the standing electromagnetic wave, is formed in the fluid pipe.

Furthermore, the invention relates to a pyrolysis method, with which a fluid with a part to be pyrolysed is guided through a pyrolysis cell which is acted upon by a standing electromagnetic wave.

Such an apparatus and such a method are known from EP 0 489 078 B1. Pyrolysis is the thermal degradation of chemical compounds (with or without oxygen access). Organic vapors, in particular, solvent vapors may, for example, be disposed of by means of such an apparatus and such a method in that the corresponding, organic molecules are pyrolysed by being acted upon by microwaves.

Proceeding on this basis, the object underlying the invention is to improve the method specified at the outset and the apparatus specified at the outset such that a high rate of degradation of molecules to be pyrolysed can be achieved.

### SUMMARY OF THE INVENTION

This object is accomplished in accordance with the invention, in the case of the apparatus specified at the outset, in that the pyrolysis cell is limited in the direction of an outlet by a metal grid.

Such a metal grid acts as a screening or shielding grid for the microwave, whereby it is possible to prevent a discharge dent expanding into the fluid pipe as discharge pipe outside an activator chamber (the pyrolysis cell). In this respect, the formation of a wave propagating in the fluid pipe is, in particular, essentially suppressed. This propagating wave would, without the metal grid, run along the fluid pipe through it and, therefore, remove energy from the pyrolysis cell. As a result, a considerable proportion of the microwave power coupled in would, on the other hand, not be available for activating molecules but, rather, would have to be used for the formation of the wave, wherein this wave then distributes this energy over a larger spatial area.

In accordance with the invention, a high energy density may, therefore, be coupled into a spatially limited area via a metal grid and so a high degree of activation prevails, accordingly, in this area in the fluid pipe. As a result, high flow rates, for example, in the order of magnitude of 5 m³ per h of a fluid through the fluid pipe may also be processed.

Test measurements have, for example, shown that fluorinated hydrocarbons, homogenized hydrocarbons and aromatic hydrocarbons may be degraded with a high rate of degradation, for example, with a rate of flow of 3 m³ per h and a concentration of 1,500 ppm with rates of degradation of greater than 98% for acetone, toluene and dichloromethane.

The degradation is brought about in that a microwave plasma is formed in the fluid within the pyrolysis cell and the corresponding molecules are dissociated and ionized in the microwave plasma due to electron collisions of the free electrons in the plasma.

It is particularly advantageous when the pyrolysis cell is limited by a first metal grid and a second metal grid arranged in spaced relationship in the fluid guidance direction. The microwave may, as a result, be concentrated on the area between these metal grids and a plasma entrainment upstream and downstream out of the pyrolysis cell is prevented to a great extent. Furthermore, high flow rates may be set since an inlet diameter, in particular, can be selected independently of the wavelength of the standing wave. (Without a shielding grid this would have to be selected to be smaller and, in particular, very much smaller than half the wavelength in order to concentrate the microwave power in the area beyond the outlet in relation to the fluid guidance direction).

In order to be able to couple a high power density into the pyrolysis cell, a wave loop of the standing electromagnetic wave is advantageously located within the pyrolysis cell.

In order to prevent as far as possible any denting of the plasma area outside the pyrolysis cell, a mesh aperture of a metal grid is advantageously smaller than half a wavelength of the standing electromagnetic wave and, in particular, smaller than 1/10 of this wavelength. As a result, the formation of a propagating wave in the pyrolysis cell may also be suppressed in an effective way.

The first metal grid and the second metal grid are advantageously aligned parallel to one another in order to obtain symmetrical conditions in the pyrolysis cell with respect to the plasma formation.

For the same reason it is advantageous when a metal grid has an essentially flat surface which is arranged, in particular, essentially at right angles to a fluid guidance direction.

Furthermore, it is favorable when a metal grid coves a free internal cross-sectional area of the fluid pipe completely in order to prevent any denting of the plasma discharge area outside the pyrolysis cell in this way.

In order to obtain an effective power coupling of the microwave power into the pyrolysis cell, an area of the waveguide passing through the fluid pipe is advantageously arranged between the first metal grid and the second metal grid, i.e., the area, in which the wave field is coupled into the pyrolysis cell, is located between the two metal grids.

It may be provided for a metal grid to be at a specific electrical potential or for a metal grid to be at a float potential. In the latter case, metal grids are then at a potential which is determined by the ion transport and the ion velocity in the fluid pipe. In this respect, it may also be provided for a specific starting potential to be applied and for floating to be allowed within a certain area.

Since great power is coupled into the pyrolysis cell, it is particularly advantageous when this is cooled by means of liquid cooling. As a result, a greater discharge of heat may be achieved than in the case of air cooling.

Silicone oil, with which a high rate of heat discharge has been able to be achieved, has proven to be a particularly suitable coolant.

In order to form suitable flow ratios and in order to enable an effective cooling, the pyrolysis cell is, in particular, of a cylindrical design.

The pyrolysis cell is surrounded by one or more annular channels as cooling channels. As a result, the pyrolysis cell may be cooled via the coolant over a large surface area.

In this respect, an annular channel is, in particular, arranged concentrically to an axis of the pyrolysis cell in order not to generate any “hot spots”. Measurements have shown that with formation of the pyrolysis cell as a quartz glass pipe, this will melt within the shortest period of time without any effective cooling.

A cooling liquid is advantageously guided through in an annular channel in counterflow to the fluid guidance direction for the effective cooling of the pyrolysis cell.

It is particularly advantageous when the fluid is guided through the fluid pipe in a turbulent flow for the purpose of convective coolability. Due to the fact that local vortices are formed in the fluid, heat may be removed from the pyrolysis cell more effectively via the cooling liquid. Comparative calculations have shown that temperatures of several thousand degrees Kelvin can be reached in the pyrolysis cell without any turbulent flow guidance. With a turbulent flow guidance, there is the additional advantage that no dead points or dead spaces can be formed in the pyrolysis cell and that external selection possibilities with respect to the dimensioning are also present.

It has proven to be favorable when the fluid is guided through the fluid pipe at a pressure of at least 30 mbar. In principle, it is also possible for the fluid to be at ambient pressure in the pyrolysis cell.

In order to facilitate an effective application of underpressure at the pyrolysis cell when acted upon by underpressure in comparison with the ambient pressure, an entry connection for fluid into the fluid pipe is advantageously provided with a smaller cross section than a corresponding exit connection for the removal of fluid.

It has proven to be favorable when, for the purpose of mineralizing toxic agents, the microwave power coupled into the waveguide is at least 3 kW. As a result, rates of degradation of 98% or greater may be achieved with a rate of flow in the order of magnitude of 3 m³ per h of hydrocarbons through the fluid pipe.

It is provided, in particular, for the waveguide to be a rectangular waveguide in order to be able to form a standing wave with a wave loop in the area of the pyrolysis cell in a simple manner.

It has proven to be favorable when the ratio of a diameter of the fluid pipe to a transverse dimensioning of the waveguide transverse to the fluid guidance direction is less than 5. As a result, sufficiently high energy densities for the purpose of forming microwave plasma and for activating molecules still result in edge areas of the fluid pipe close to the wall and, therefore, in the pyrolysis cell. These ratios can, in this respect, be scaled upwards, i.e., when a greater diameter of the fluid pipe is intended to be selected in order to bring about a greater volume rate of flow, the transverse dimensioning of the waveguide should be adapted thereto accordingly in the same way.

It is particularly advantageous when an aftercooling section is provided to follow the pyrolysis cell in a fluid guidance direction. In principle, it is possible for activated or rather dissociated molecules to recombine again and, as a result, for the process of degradation to be partially reversed again. In addition, additional toxic agents, such as dioxins and furans, can result in the case of such recombinations. If, however, an aftercooling section follows, in which the fluid which has passed through the pyrolysis cell is cooled quickly, such recombinations may be prevented to a great extent, i.e., the rate of degradation of the pyrolysis cell corresponds essentially to the rate of degradation which is achieved by the entire apparatus.

In this respect, the aftercooling section advantageously comprises a cooling system independent of the cooling of the pyrolysis cell so that pyrolysis cell and the aftercooling section can be controlled independently of one another.

The aftercooling section is, in particular, water-cooled in order to bring about a quick cooling.

In this respect, it is possible to use the aftercooling section as a reaction chamber, in which molecules activated in the pyrolysis cell can be used as reactants. The temperature in the reaction chamber may be controlled via the cooling system of the aftercooling section and, therefore, reaction processes with the reactants may be controlled.

For this purpose, one or more coupling-in connections, via which co-reactants can be introduced, are advantageously provided in the area of the aftercooling section. As a result, syntheses may be carried out, for example, with the aid of the activated molecules as reactants, wherein the corresponding co-reactants are made available via the coupling-in connections.

The waveguide can advantageously be adjusted so that a standing electromagnetic wave of a specific wavelength can be formed. Such an adjustment may be achieved via a slide which forms a limiting wall of the waveguide.

It has proven to be favorable when the frequency of the electromagnetic wave is in the range of between 0.5 GHz and 5 GHz.

The object specified at the outset is accomplished in accordance with the invention, in the pyrolysis method specified at the outset, in that the fluid is guided through the pyrolysis cell in a turbulent flow for the convective cooling thereof.

In the case of guidance in a turbulent flow, i.e., in the case of the adjustment of Reynolds numbers which are greater than approximately 2,300 and, in particular, greater than 10,000, it is provided for a cooling liquid to be able to remove heat from limiting walls of the pyrolysis cell effectively.

In addition, the formation of dead points and dead spaces in the pyrolysis cell can be prevented via the formation of a turbulent flow and greater selection possibilities with respect to the dimensioning of the apparatus for carrying out the method result.

Additional, advantageous developments of the inventive method have already been explained in conjunction with the inventive apparatus.

### BRIEF DESCRIPTION OF THE DRAWINGS

The following description of preferred embodiments serves to explain the invention in greater detail in conjunction with the drawings. These show:

FIG. 1 a perspective view of an inventive pyrolysis apparatus in an exploded illustration;

FIG. 2 a lateral sectional view through a fluid pipe;

FIG. 3 a schematic cross-sectional view through the fluid pipe according to FIG. 2 at right angles to its plane of drawing;

FIG. 4 an illustration of the spatial distribution of the field strength parts Eₓ, E_{y} in a waveguide and in the fluid pipe;

FIG. 5 the same view as in FIG. 4, wherein the distribution of the field strength component E_{z} is shown and dots designate field strength vectors which lead out of the plane of drawing and crosses, with which the direction is into the plane of drawing;

FIG. 6 a schematic view of a fluid pipe with activator chamber and reaction chamber;

FIG. 7 an instantaneous representation in time of an excitation wave propagating in the fluid pipe when no metal grid is provided, and

FIG. 8 measurement results of rates of degradation via the microwave power which have been obtained by means of an inventive apparatus from a mixture of dichloromethane and air with a proportion of dichloromethane of 1,600 ppm; the different curves belong to different pressures and rates of flow.

### DETAILED DESCRIPTION

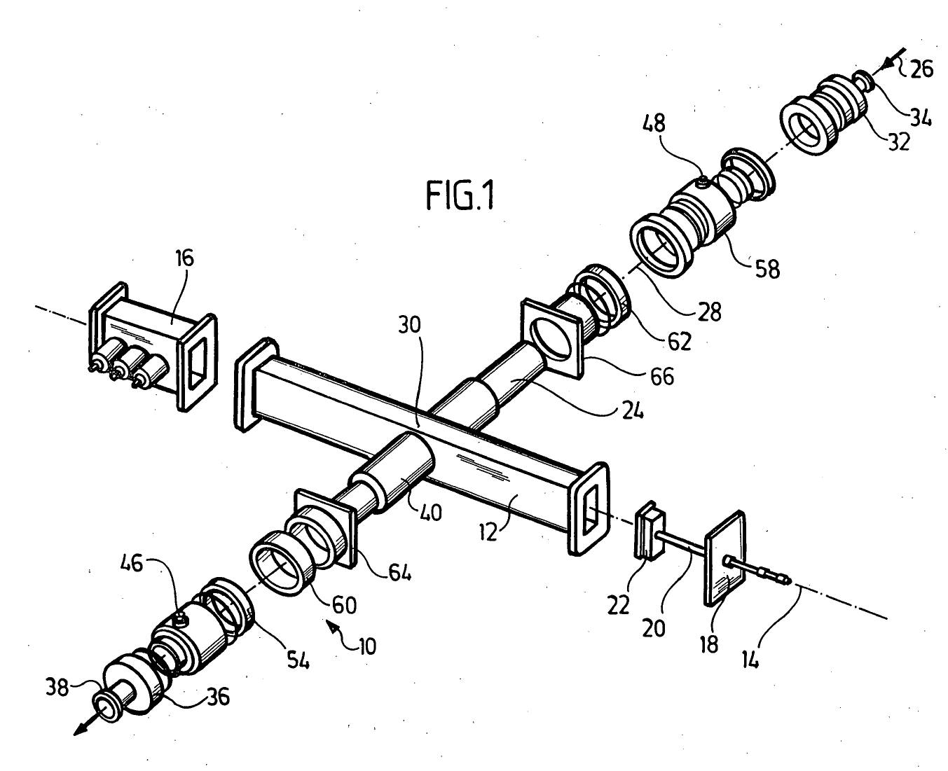

In one embodiment of an inventive pyrolysis apparatus which is designated in FIG. 1 as a whole as 10, a rectangular waveguide 12 is coupled to a microwave generator (not shown in the drawings) which generates microwaves. Standing waves may be formed in the waveguide 12 with a direction of propagation parallel to a longitudinal direction 14 of the waveguide 12. In particular, standing waves in the S band may be formed.

The microwave generator supplies microwaves in the frequency range, for example, of between 0.5 GHz and 5 GHz.

It may be provided for a reflection measurement cell 16 to be arranged at one end of the waveguide 12 in order measure the reflections. With its aid, the coupling of the microwave power into the waveguide 12 may also be optimized; the microwave generator is then arranged so as to be connected to the output of the reflection measuring cell 16.

A bearing plate 18 is seated at the other end of the waveguide 12 and a spindle 20, which, on the other hand, holds a waveguide wall 22, is mounted for displacement in this bearing plate. By displacing this spindle 20 relative to the bearing plate 18, the distance of the waveguide wall 22 to just this bearing plate 18 may be adjusted in order to be able to carry out in this way an adaptation of the waveguide limiting surfaces effective for the formation of a standing electromagnetic wave. In particular, the waveguide wall 22 may be displaced such that the standing wave is formed at a given frequency and, in addition, a wave loop as area of greatest radiation density is located where a fluid is guided through the waveguide 12.

A fluid may be carried through by means of the electromagnetic wave field which can be formed in the waveguide 12. For this purpose, a fluid pipe 24 is guided through the waveguide 12, namely, in particular, in a central area of the waveguide 12 with a fluid guidance direction 26 transverse and, in particular, at right angles to the longitudinal direction 14 of the waveguide 12.

The fluid pipe 24 is, for this purpose, of a cylindrical design with an axis 28 which passes preferably centrally through the waveguide 12 with respect to a direction transverse to the longitudinal direction 14. The fluid pipe 24 is, for example, produced from a quartz glass in order to ensure the transparency of the microwaves in addition to a high heat resistance.

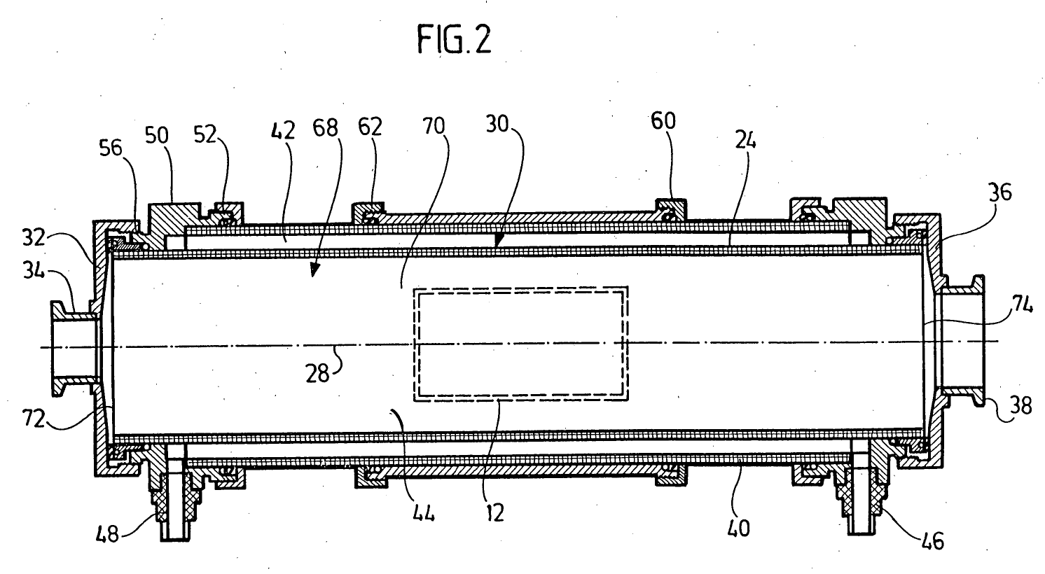

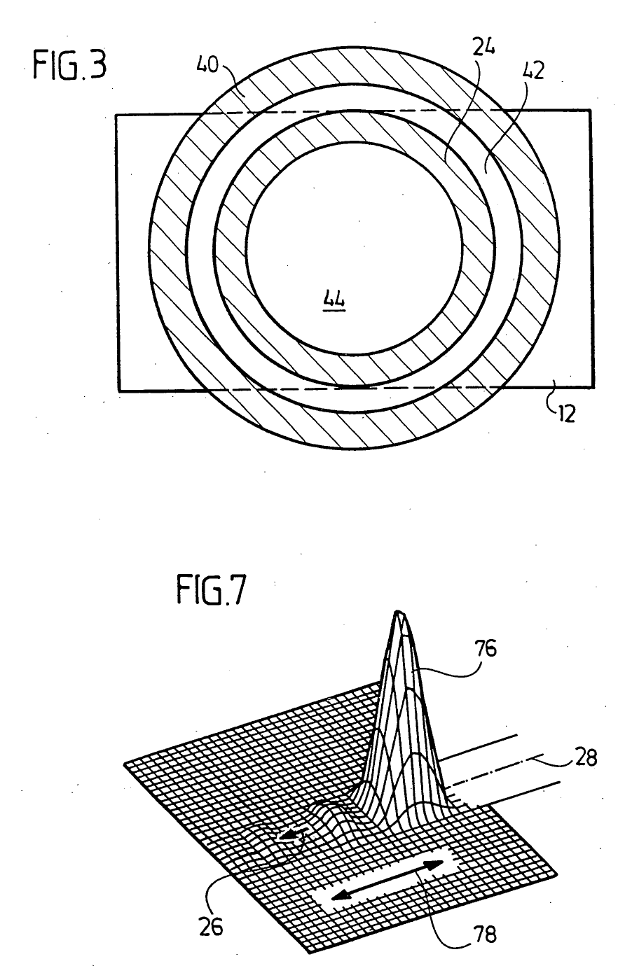

That area 30 of the fluid pipe 24 (cf. FIG. 2) which is guided through the waveguide 12 can be acted upon, as a result, with electromagnetic radiation energy.

The fluid pipe 24 is provided with an entry flange 32 which has a short connection pipe 34, via which a fluid can be coupled into the fluid pipe 24. For this purpose, a corresponding fluid line can, in particular, be coupled to the short connection pipe 34.

At the opposite end, the fluid pipe is provided with an exit flange 36 with a short connection pipe 38, via which the fluid which has flowed through the fluid pipe can be discharged from it.

In this respect, the short connection pipe 34 preferably has a smaller cross section than the short connection pipe 38 for the exit in order to be able to maintain underpressure better at the short connection pipe 38 when fluid is guided through by means of a pump in order to ensure in this way that the fluid which has been coupled into the fluid pipe 24 is again discharged from it.

The fluid pipe 24 forms an inner pipe which is surrounded by an outer pipe 40 which is likewise of a cylindrical design with an axis which coincides with the axis 28 of the fluid pipe 24. This outer pipe 40 is likewise produced, for example, from quartz glass. An annular chamber 42 is formed between the outer pipe 40 and the fluid pipe 24 and this acts as a coolant channel, through which a coolant can be guided, with which the fluid pipe 24 can be cooled in order to be able to remove heat from an interior chamber 44 of the fluid pipe 24.

In this respect, an inlet connection 46 is provided for forming a counterflow cooling, wherein this inlet connection is located in the vicinity of the exit flange 36. A coolant and, in particular, a cooling liquid, may be introduced into the annular chamber 42 via this inlet connection 46, wherein this coolant then flows through the annular chamber in a direction contrary to the fluid guidance direction 26.

The coolant heated accordingly may be discharged via an outlet connection 48 which is arranged in the vicinity of the entry flange 32.

In accordance with the invention, a cooling liquid, such as silicone oil, with a high heat absorption capacity is used in order to achieve a high cooling effect. For example, silicone oil is pumped through the annular chamber 42 with a volume flow of 10 l per minute.

In order to connect and keep a space between the fluid pipe 24 as inner pipe and the outer pipe 40, a first connecting device 50 is provided in the area of the entry flange 32 and this is held at one end 52 on a, for example, ring-shaped flange 54 which is connected to the outer pipe 40 and is held at another end 56 on the fluid pipe 24, for example, by means of the entry flange 32.

A corresponding, second connecting device 58, which is, in particular, of the same design as the first connecting device 50, holds the outer pipe 40 relative to the inner pipe 24 in the area of the exit flange 36.

In addition, it may be provided for the outer pipe 40 to hold spaced annular flanges 60, 62, via which the outer pipe 40 can be fixed relative to the waveguide 12 by means of additional, respectively associated holding devices 64, 66 in order to provide for an additional hold in this way.

A pyrolysis cell 68 with an activator chamber 70 is formed in the interior 44 of the fluid pipe 24. Chemical compounds may be thermally decomposed in this pyrolysis cell 68 (with or without oxygen access) in that molecules are excited by means of electron impact processes via a microwave plasma generated by the standing wave. These electron impact processes in the plasma cause dissociation and ionization of molecules or ionization without previous dissociation.

The pyrolysis cell 68 is, in relation to the fluid guidance direction 26, closed “electrically” by means of a first metal grid 72 and a second metal grid 74 arranged in spaced relationship thereto (cf. FIG. 2), wherein the flow of fluid through the metal grids 72 and 74 is essentially not hindered. The mesh aperture of the metal grids 72, 74 is smaller than half the wavelength of the standing wave in the waveguide 12 and, in particular, smaller than 1/10 of this wavelength. As a result, a microwave shielding grid is formed each time which prevents the formation of a discharge dent in areas with a lower electromagnetic energy density. As a result, the area, in which the microwave plasma is formed, may be limited to the area between the two metal grids 72 and 74 and, therefore, the energy coupling into the fluid for the formation of the microwave plasma may be limited accordingly. As a result, a high power density may be coupled into the fluid since the metal grids 72 and 74 limit the activator chamber 70.

The metal grids 72 and 74 are aligned parallel to one another with a surface normal essentially parallel to the axis 28.

The metal grids 72 and 74 are, in particular, seated in the fluid pipe 24 such that the waveguide 12 is arranged between them, i.e., that, as a result, a wave loop of the standing electromagnetic wave in the waveguide 12 is also located between the metal grids 72 and 74.

To what extent a discharge is dented beyond the metal grids 72 and 74 away from the waveguide 12 may be adjusted via the mesh aperture selected.

It may be provided for the metal grids 72 and 74 to each be at a specific electrical potential or be at float potentials, i.e., be at an electrical potential which is determined by ion transport and ion velocity in the fluid pipe 24.

The entrainment of the microwave plasma in the fluid pipe 24 upstream and downstream in the fluid guidance direction 26 is prevented by the metal grids 72 and 74 so that a high energy coupling into the activator chamber 70 of the pyrolysis cell 68 is achieved as a result.

An instantaneous photograph of an excitation wave 76 in the pyrolysis cell 68 is shown in FIG. 7, wherein this wave has a wavelength 78; the diagram shows the results of a simulation as intensity over the plane of the axis 28 (x-y coordinates) without the activator chamber 70 being limited by the shielding metal grids 72, 74. As a consequence, as shown in FIG. 7, a wave is formed with a vector of propagation parallel to the fluid guidance direction 26 and this removes, to a certain extent, energy density from the fluid pipe 24 which is, therefore, no longer available for the purpose of exciting fluid in the fluid pipe 24.

The formation of such a wave 76 is suppressed to a great extent due to the arrangement of the metal grids 72 and 74.

The inventive pyrolysis method functions as follows:

A standing electromagnetic wave is generated in the waveguide 12 with a loop which is located in the area, in which the fluid is guided through the fluid pipe 24 through the waveguide 12, i.e., in the pyrolysis cell 68. The microwave generator thereby supplies power which is at least 3 kW. The microwave frequency is, for example, 2.45 GHz.

A fluid is guided through the activator chamber 70 as pyrolysis cell 68 with a pressure which is, for example, in the range between 20 mbar and 1,000 mbar. In the case of an air mixture, a self-ignition has, for example, occurred at an entry pressure of 20 mbar.

A microwave plasma is formed in the fluid, which flows through the fluid pipe 24, within the activator chamber 70. Molecules are, on the other hand, activated in the fluid via dissociation and/or ionization due to electron impact processes of the free electrons in the microwave plasma. When the fluid represents a mixture consisting of a carrier fluid and, for example, organic toxic agents which are to be mineralized, these organic toxic agents, for example, chlorinated hydrocarbon or fluorinated hydrocarbon may be degraded as a result and disintegrated into less toxic substances or rather substances which are easier to process, such as CO, CO₂, Nₓ, H₂O or HCl.

Due to the fact that a high energy density is present in the activator chamber 70, an effective cooling must be provided. This is brought about via a liquid cooling via the annular chamber 42 in accordance with a counterflow principle, i.e., the coolant is guided through via the inlet connection 46 in the direction of the outlet connection 48 in a direction opposite to the fluid guidance direction 26. In order to obtain an effective cooling, a convective principle is used, i.e., the fluid is guided through the fluid pipe 24 in a turbulent flow in order to obtain a local vortex motion in the fluid. Local flow vortices, via which fluid impinges on limiting walls of the fluid pipe 24, are then formed, wherein heat can, on the other hand, be removed from them via the silicone oil in the annular chamber 42. For example, Reynolds numbers greater than 10,000 have proven to be advantageous for an effective, convective cooling of the fluid pipe 24.

As a result of a turbulent flow of the fluid in the fluid pipe 24, it is also possible for essentially no dead points to be formed in the activator chamber 70, in which toxic agents could collect. In addition, greater selection possibilities for the dimensioning also exist.

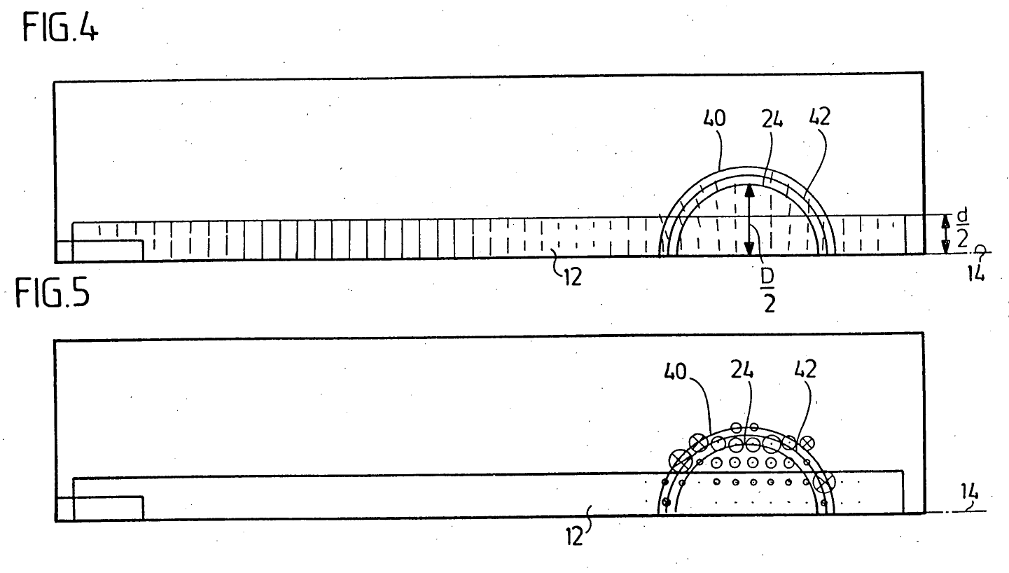

FIGS. 4 and 5 show by way of example the distribution of the electrical field strength of the standing electromagnetic wave in the waveguide 12 and in the fluid pipe 24, wherein the components Eₓ, E_{y} are shown in FIG. 4 and the component E_{z} in FIG. 5 at right angles thereto.

In FIG. 4, the length of the dashes indicates the field strength, wherein it is apparent that a wave loop is formed in the area of the fluid pipe 24 so that a high energy density can be coupled in. The field strength is still sufficiently great in the outer area of the fluid pipe 24, i.e., in the area of the vicinity of the annular chamber 42 for a high energy density to prevail in order to excite a microwave plasma accordingly and, as a result, to be able to activate molecules over the entire cross section of the fluid pipe 24.

It has been shown that, when the ratio of a diameter D of the fluid pipe 24 to a corresponding transverse dimension d of the waveguide 12 is less than approximately 5 and, in particular, less than approximately 3, a sufficiently large energy density is still present even in the edge areas of the fluid pipe 24.

This ratio can be scaled, i.e., with a corresponding increase in the diameter D of the fluid pipe 24 the corresponding ratios can be obtained by means of an increase in the same way of the transverse dimension d of the waveguide 12.

If, for example, a greater volume flow through a fluid pipe 24 is intended to be adjusted and, consequently, the diameter of the fluid pipe 24 adapted, an effective coupling of energy into the fluid pipe 24 can still be ensured by an increase in the size of the waveguide 12 in the same way.

The shielding metal grids 72 and 74 have the effect that the formation of a propagating wave 76, which could, in particular, remove energy from the fluid pipe 24 downstream in relation to the fluid guidance direction 26, is essentially suppressed.

The energy coupled into the activator chamber 70 is then available with a high effectiveness for the formation for the microwave plasma and, accordingly, for the activation of the corresponding molecules in the fluid.

Advantageously, the fluid discharged from the activator chamber 70 via the exit flange 36 is cooled so that no recombination of the activated molecules can take place in order to prevent any renewed formation of “initial molecules” to a great extent. In this respect, a fast cooling process takes place, in particular.

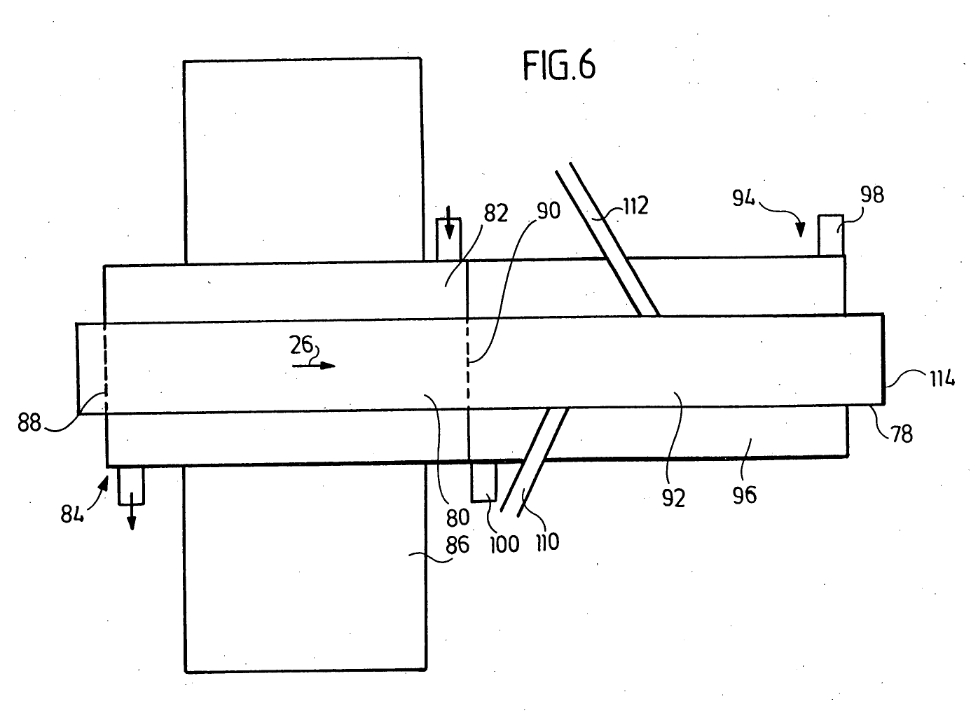

FIG. 6 shows schematically a variation of one embodiment, with which an activator chamber 80 is formed within a fluid pipe 78, this activator chamber being, in principle, of the same design and functioning as described above. This activator chamber 80 can, in particular, be cooled with a liquid and, in particular, silicone oil via an annular chamber 82. A corresponding cooling system is designated in FIG. 6 as a whole as 84.

The activator chamber 80 is guided through a waveguide 86, in which a standing wave can be formed, as described above, with a wave loop in the activator chamber 80.

The activator chamber 80 is, on the other hand, limited by a first metal grid 88 and a second metal grid 90 which, again, have the same function as described above on the basis of the metal grids 72 and 74.

The second metal grid 90 separates the activator chamber 70 from a reaction chamber 92 formed in the fluid pipe 24. This reaction chamber 92 has a cooling system 94 which is independent of the cooling system 84 of the activator chamber 80.

The temperature of the reaction chamber 92 may be controlled via this cooling system 94 which comprises an annular chamber 96 which surrounds the reaction chamber 92 and through which a coolant can be conducted in a counterflow direction to the fluid guidance direction 26.

The coolant, in particular, water can be introduced into the annular chamber 96 via a connection 98 and the coolant may be discharged via a connection 100.

The reaction chamber 92 can also be arranged outside the fluid pipe 78 in that, for example, a corresponding pipe is post-connected to the fluid pipe 24 in order to form a reaction chamber.

In accordance with the invention, a rapid cooling process may be carried out in the activated fluid by means of the reaction chamber 92 post-connected to the activator chamber 80 in order to prevent any reformation of, for example, organic molecules following the dissociation. As a result, a corresponding recombination can be prevented which could also lead to the formation of toxic agents, such as dioxins or furans.

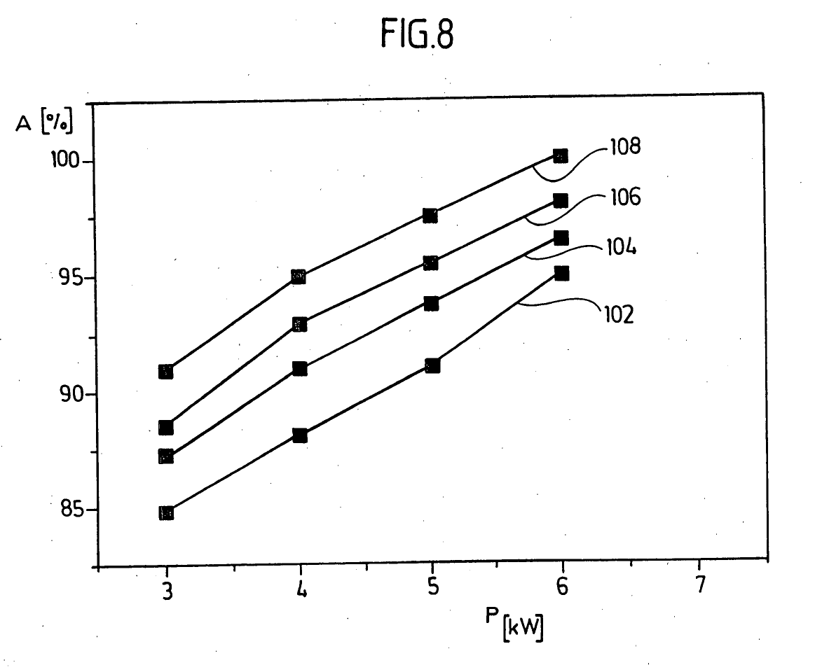

In FIG. 8, an example of a measurement with the inventive pyrolysis apparatus according to the inventive pyrolysis method is shown, wherein a specific volume flow of a mixture of air with 1,600 ppm of dichloromethane (this corresponds to the saturation value of dichloromethane-air mixtures) has been guided through the fluid pipe 24. The rate of degradation is shown in percent (100% means a complete degradation of dichloromethane) over the microwave power. During the measurements, the maximum achievable power of the microwave generator was 6.0 kW at a frequency of 2.45 GHz. The curve 102 shows a flow rate of 6,000 l per h at a pressure of 80 mbar in the fluid pipe 24. The curve 104 shows the measured values at a flow rate of 5,000 l per h at a pressure of 70 mbar, the curve 106 a flow rate of 4,000 l per h at a pressure of 60 mbar and, finally, the curve 108 a flow rate of 3,000 l per h at a pressure of 50 mbar.

As is apparent from these measured data, a high rate of degradation can be achieved even with high flow rates by a corresponding increase in the coupling-in of microwave power, this rate of degradation being almost 100% with a sufficiently high coupling-in of power. In principle, the result of degradation can be improved with a predetermined rate of flow by increasing the microwave power. The inventive apparatus and the inventive method may, therefore, be adapted to the respective application, i.e., the desired degradation result may be achieved, depending on the rate of flow, via adjustment of pressure and adjustment of power.

The recombination of the dissociated molecules may be prevented by a rapid cooling in the reaction chamber 92.

Measurements have also shown that with a flow rate of 3,000 l per h and a concentration of 1,500 ppm for acetone, a rate of degradation of greater than 99% can be achieved, for toluene likewise greater than 99% and for dichloromethane greater than 98%, as shown in FIG. 8.

The inventive apparatus may be designed as a “table device”, i.e., it is transportable. It is fundamentally possible by means of the inventive apparatus to operate in the activator chambers 70 and 80, respectively, with atmospheric pressure so that transportability is also ensured as a result.

Organic solvents can, for example, be decomposed by means of the inventive pyrolysis apparatus and the inventive pyrolysis method and, therefore, a method and an apparatus are made available for the disposal of organic vapors, in particular, of solvent vapors.

The inventive apparatus may also be used as a microwave reactor, in particular, for carrying out a method for the control of the reaction of activated molecules. The molecules activated in the activator chamber 80 pass into the reaction chamber 92 and represent, in principle, reactants. They may react with additional molecules, wherein the reaction conditions can be adjusted. On the one hand, the type of activation in the activator chamber 80 may be adjusted to a certain degree. The reaction conditions may be adjusted in the reaction chamber 92 by means of temperature control via the cooling system 94 for the reaction chamber. The activated molecules then acting as reactants may be used accordingly. A possible application is, for example, the production of acetic acid or ammonia synthesis. In the latter case, nitrogen is then excited in the activator chamber 80 and hydrogen is introduced into the reaction chamber 92 as co-reactant.

In a variation of one embodiment it is provided for co-reactants for the reactants—the activated molecules—to be supplied to the reaction chamber 92 via one or more supply lines 110, 112, each of which opens into the reaction chamber.

The products of reaction can then be discharged via an exit 114.

It should now be appreciated that the present invention provides advantageous methods and apparatus for pyrolysing a fluid.

Although the invention has been described in connection with various illustrated embodiments, numerous modifications and adaptations may be made thereto without departing from the spirit and scope of the invention as set forth in the claims.

What is claimed is:

1. Pyrolysis method for pyrolysing a fluid, comprising;
generating a standing electromagnetic wave;
guiding a fluid to be pyrolysed through a fluid pipe in a fluid guidance direction transversely to a direction of propagation of the standing electromagnetic wave;
forming a microwave plasma in the fluid within a pyrolysis cell of the fluid pipe and disassociating corresponding molecules of the fluid as a result of the fluid being acted upon in the pyrolysis cell by the standing electromagnetic wave; and
preventing formation of a discharge dent in areas of the fluid pipe having a low electromagnetic energy density;
wherein:
said areas of the fluid pipe having a low electromagnetic energy density are outside of said pyrolysis cell; and
the formation of the discharge dent outside of the pyrolysis cell is prevented by arranging a first metal grid and a second metal grid in spaced relationship in the fluid guidance direction in the pyrolysis cell, a mesh aperture of the first metal grid and the second metal grid being smaller than half a wavelength of the standing electromagnetic wave.

2. Pyrolysis method in accordance with claim 1, wherein a wave loop of the standing electromagnetic wave is located within the pyrolysis cell.

3. Pyrolysis method in accordance with claim 1, wherein the first metal grid and the second metal grid are aligned parallel to one another.

4. Pyrolysis method in accordance with claim 1, wherein the metal grids have an essentially flat surface.

5. Pyrolysis method in accordance with claim 4, wherein the metal grids are arranged essentially at right angles to said fluid guidance direction.

6. Pyrolysis method in accordance with claim 1, wherein the metal grids each cover a free internal cross-sectional area of the fluid pipe completely.

7. Pyrolysis method in accordance with claim 1, wherein the standing electromagnetic wave is generated in a waveguide by a microwave generator coupled to said waveguide.

8. Pyrolysis method in accordance with claim 7, wherein an area of the waveguide passes through the fluid pipe and is located between the first metal grid and the second metal grid.

9. Pyrolysis method in accordance with claim 7, wherein at least 3 kW of microwave power is coupled into the waveguide for the purpose of mineralizing toxic agents.

10. Pyrolysis method in accordance with claim 7, wherein the waveguide is a rectangular waveguide.

11. Pyrolysis method in accordance with claim 7, wherein the ratio of a diameter of the fluid pipe to a transverse dimension of the waveguide transverse to the fluid guidance direction is less than five to 1.

12. Pyrolysis method in accordance with claim 7, wherein the waveguide is adjustable so that a standing electromagnetic wave of a certain wavelength is able to be formed.

13. Pyrolysis method in accordance with claim 1, wherein the metal grid is at a specific electrical potential.

14. Pyrolysis method in accordance with claim 1, wherein the metal grid is at a float potential.

15. Pyrolysis method in accordance with claim 1, wherein the pyrolysis cell is cooled by way of liquid cooling.

16. Pyrolysis method in accordance with claim 15, wherein silicone oil is used as coolant.

17. Pyrolysis method in accordance with claim 1, wherein the pyrolysis cell is of a cylindrical design.

18. Pyrolysis method in accordance with claim 17, wherein the pyrolysis cell is surrounded by one or more annular channels as cooling channels.

19. Pyrolysis method in accordance with claim 18, wherein said annular channel is arranged concentrically to an axis of the pyrolysis cell.

20. Pyrolysis method in accordance with claim 18, wherein a cooling liquid is guided through in said annular channel in counterflow to the fluid guidance direction.

21. Pyrolysis method in accordance with claim 1, wherein the fluid is guided in a turbulent flow through the fluid pipe for the purpose of convective coolability.

22. Pyrolysis method in accordance with claim 1, wherein the fluid is guided through the fluid pipe at a pressure of at least 30 mbar.

23. Pyrolysis method in accordance with claim 1, wherein an entry connection for fluid into the fluid pipe has a smaller cross section than an exit connection.

24. Pyrolysis method in accordance with claim 1, wherein an aftercooling section following the pyrolysis cell in said fluid guidance direction is provided.

25. Pyrolysis method in accordance with claim 24, wherein the aftercooling section comprises a cooling system independent of the cooling of the pyrolysis cell.

26. Pyrolysis method in accordance with claim 25, wherein the aftercooling section is water-cooled.

27. Pyrolysis method in accordance with claim 24, wherein the aftercooling section is usable as a reaction chamber, molecules activated in the pyrolysis cell being usable as reactants in said reaction chamber.

28. Pyrolysis method in accordance with claim 24, wherein one or more coupling-in connections are provided in an area of the aftercooling section.

29. Pyrolysis method in accordance with claim 1, wherein the frequency of the electromagnetic wave is in the range of between 0.5 GHz and 5 GHz.
